(19) United States
(12) Patent Application Publication
(10) Pub. No.: US 2007/0049569 A1
(43) Pub. Date: Mar. 1, 2007

(54) Methods of using fatty-acid esters of estrogens and thermogenic compounds for reducing the body weight of a mammal and compositions containing the same

(75) Inventor: Maria Alemany, Barcelona (ES)

Correspondence Address: JONES DAY, 222 EAST 41ST ST, NEW YORK, NY, 10017, US

(73) Assignee: Oleoyl-Estrone Developments SL

(21) Appl. No.: 11/472,694

(22) Filed: Jun. 21, 2006

Related U.S. Application Data
(62) Division of application No. 10/969,249, filed on Oct. 18, 2004.

(51) Int. Cl. A61K 31/56; A61K 31/202

(52) U.S. Cl. 514/171; 514/182; 514/560

(57) ABSTRACT

Compositions and methods for reducing the body weight of a mammal are disclosed. The invention is directed to methods for reducing the body weight in a mammal comprising administering therapeutically effective amounts of a fatty-acid ester of an estrogen or estrogen derivative and a fatty-acid, and a thermogenic compound. Furthermore the invention is directed to compositions comprising a fatty-acid ester of an estrogen or estrogen derivative and a fatty-acid, and a thermogenic compound.

### FIELD OF THE INVENTION

This invention relates generally to compositions and methods for reducing the body weight of a mammal. More particularly, the invention is directed to methods for reducing the body weight in a mammal comprising administering therapeutically effective amounts of a fatty-acid ester of an estrogen or estrogen derivative and a fatty-acid, and a thermogenic compound. Furthermore the invention is directed to compositions comprising a fatty-acid ester of an estrogen or estrogen derivative and a fatty-acid, and a thermogenic compound.

### BACKGROUND OF THE INVENTION

Treatment of obesity and/or overweight is a therapeutic or cosmetic problem of major importance that does not have a satisfactory solution yet. Attempts to solve the problem by physical exercise or reduction of food intake, are well known. But also known are the difficulties, limitations and general lack of success of all these approaches. Apparently the sheer complexity of mechanisms involved in the control of body mass allow little room for external manipulation, thus limiting the possible damage to body reserves by increased thermogenic stimulation or diminished energy intake.

In the therapeutic fight against obesity and/or overweight, considerable research has focused on trying to find a signal that informs the brain of the size of fat tissue mass. It is believed that such information is required by the brain to promote either the accumulation of fat reserves or their burning by the thermogenic system, via the natural homeostatic mechanisms set to maintain the body mass stable.

Estradiol or estra-1,3,5(10)-triene-3,17-diol is a natural estrogen widely used in estrogenic hormone therapy. Estradiol esters at C-17 and C-3 with palmitic, stearic, and oleic acids have been chemically synthesized and their long-term estrogenic responses in ovariectomized rats have been reported (cf. M. A. Vazquez-Alcantara et al., J. Steroid Biochem. 33:1111-18 (1989)). However, nothing was suggested about the use of these estradiol esters in the treatment of obesity.

Alemany disclosed fatty-acid esters of estrogens for the treatment of obesity and overweight in U.S. Pat. No. 5,798,348, which is incorporated herein by reference in its entirety for all purposes. For instance, the fatty-acid monoester estrone monooleate (“oleoyl-estrone”), a powerful slimming agent for the treatment of obesity and overweight with marked hypolipemic and antidiabetic effects, was studied. Oleoyl-estrone acts as a ponderostat signal informing the body weight control system of the mass of fat reserves held in the body. In the morbidly obese, this signaling is altered. Its main physiological effects are a decrease in voluntary food intake with maintenance of energy expenditure, which creates an energy gap that is fulfilled at the expense of lipid reserves, thus decreasing the body fat mass, sparing protein, largely decreasing the need for carbohydrate and reducing insulin resistance. Furthermore, Girouard in PCT Publication Nos. WO03/018529 and WO2004/045560, which are incorporated by reference herein in their entirety for all purposes, discloses additional fatty-acid esters of estrogens or estrogen derivatives and fatty-acids for treating obesity.

Also, a number of adrenergic agonists have been used, in the treatment of obesity and overweight. Stimulation of noradrenergic pathways, both central and peripheral, results in increased thermogenesis and energy expenditure. Adrenergic agonists enhance the energy demands of the body and thus facilitate the disposal of unwanted fat reserves.

However, overall adrenergic stimulation has serious drawbacks. In addition to increased energy expenditure, there are undesired effects, such as increases in heart rate and arterial pressure. The discovery of atypic beta-3 adrenergic receptors in brown and white adipose tissues prompted the development of a number of specific beta-3 agonists that may induce increases in lipolysis and thermogenesis without unwanted general adrenergic stimulation. In spite of the obvious advantages of such compositions, no beta-3 adrenergic agonists are available yet for human use, because of rapid loss of effectivity due to down-regulation of the receptors and marked interspecific differences in the structure and regulation of the receptors. The experimental drug CL-3 16243 is an example of a beta-3 adrenergic agonist widely used on rats and experimentally in humans.

Since it is of great interest to provide satisfactory new products for the treatment of obesity and/or overweight, the present invention relates generally to methods and/or compositions for reducing the body weight of a mammal. More particularly, the invention is directed to methods for reducing the body weight in a mammal comprising administering therapeutically effective amounts of a fatty-acid ester of an estrogen or estrogen derivative and a fatty-acid, and a thermogenic compound. Furthermore the invention is directed to compositions comprising a fatty-acid ester of an estrogen or estrogen derivative and a fatty-acid, and a thermogenic compound.

### SUMMARY OF THE INVENTION

In this specification, the term “estrogen” refers to the substances tending to promote estrus and stimulate the development of female secondary sex characteristics. This term comprises natural, semisynthetic and synthetic estrogens, both steroidal and nonsteroidal, such as estrone, diethylstilbestrol, estriol, estradiol and ethinyl estradiol. The term “estrogen derivative” refers to a compound that is derived from estrogen and usually maintains its general structure. Estrogen and estrogen derivatives are substances that induce biological responses linked to the stimulation of estrogen receptors and other biological systems that result in biological actions similar to those of estradiol and estrone. In this specification, the term “fatty acids” refers to the carboxylic acids that are components of natural fats, such as oleic acid, linoleic acid, linolenic acid, stearic acid, palmitic acid, palmitoleic acid, arachidonic acid, eicosenoic acid, docosenoic acid, and tetracosenoic acid. The term “thermogenic compounds” includes adrenergic agonists, which enhance the energy demands of the body and thus facilitate the disposal of unwanted fat reserves, such as but not limited to beta-3-agonists. In general, a thermogenic compound induces an increase in energy expenditure even in the absence of physical work. In other words, thermogenesis comprises the elimination of excess energy through increased energetic inefficiency.

In one embodiment, the invention is directed to a method for reducing body weight in a mammal. The method comprises administering a therapeutically effective amount of a fatty-acid ester of an estrogen or an estrogen derivative and a fatty acid; and administering a therapeutically effective amount of a thermogenic compound.

In another embodiment, the method for reducing body weight in a mammal comprises administering a therapeutically effective amount of a fatty-acid monoester of an estrogen or an estrogen derivative and a fatty acid and administering a therapeutically effective amount of a thermogenic compound. The estrogen can comprise estrone, diethylstilbestrol, estriol or ethinyl estradiol. Also, the fatty-acid can comprise oleic acid, linoleic acid, linolenic acid, stearic acid, palmitic acid, palmitoleic acid, arachidonic acid, eicosenoic acid, docosenoic acid, or tetracosenoic acid. Moreover, when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.

In another embodiment, the method for reducing body weight in a mammal comprises administering a therapeutically effective amount of the fatty-acid monoester oleoyl-estrone; and administering a therapeutically effective amount of a beta-3 adrenergic agonist, such as, for example, CL-316243.

Also, in one embodiment, the method for reducing body weight in a mammal comprises administering a first composition comprising a therapeutically effective amount of a fatty-acid ester of an estrogen or an estrogen derivative and a fatty acid; and administering a second composition comprising a therapeutically effective amount of a thermogenic compound.

In another embodiment, the method for reducing body weight in a mammal comprises administering a therapeutically effective amount of a first composition comprising a substantially pure fatty-acid monoester of an estrogen or an estrogen derivative and a fatty acid, and administering a second composition comprising a therapeutically effective amount of a thermogenic compound. The estrogen can comprise estrone, diethylstilbestrol, estriol or ethinyl estradiol; and the fatty acid can comprise oleic acid, linoleic acid, linolenic acid, stearic acid, palmitic acid, palmitoleic acid, arachidonic acid, an eicosenoic acid, a docosenoic acid, or a tetracosenoic acid. Also, when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.

In yet another embodiment, the method for reducing body weight in a mammal comprises administering a first composition comprising a therapeutically effective amount of oleoyl-estrone; and administering a second composition comprising a therapeutically effective amount of a beta-3 adrenergic agonist, such as, for example, CL-316243.

Furthermore, in an embodiment, the invention relates to a composition for reducing body weight in a mammal comprising (a) a therapeutically effective amount of a fatty-acid ester of an estrogen or an estrogen derivative and a fatty acid; and (b) a therapeutically effective amount of a thermogenic compound.

In another embodiment, the composition for reducing body weight in a mammal comprises a therapeutically effective amount of a substantially pure fatty-acid monoester of an estrogen or an estrogen derivative and a fatty acid, and a therapeutically effective amount of a thermogenic compound. The estrogen comprises estrone, diethylstilbestrol, estriol or ethinyl estradiol; and the fatty acid comprises oleic acid, linoleic acid, linolenic acid, stearic acid, palmitic acid, palmitoleic acid, arachidonic acid, eicosenoic acid, docosenoic acid, or tetracosenoic acid. Also, when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.

In another embodiment, the composition for reducing body weight in a mammal comprises a therapeutically effective amount of oleoyl-estrone and a therapeutically effective amount of a beta-3 adrenergic agonist, such as, for example, CL-3 16243.

Moreover, in one embodiment, the invention is directed to a method for reducing body weight in a mammal comprising administering a composition comprising (a) a therapeutically effective amount of a fatty-acid ester of an estrogen or an estrogen derivative and a fatty acid; and (b) a therapeutically effective amount of a thermogenic compound.

In another embodiment, the method for reducing body weight in a mammal comprising administering a composition comprising (a) a therapeutically effective amount of a substantially pure fatty-acid monoester of an estrogen or an estrogen derivative and a fatty acid, and (b) a therapeutically effective amount of a thermogenic compound. The estrogen comprises estrone, diethylstilbestrol, estriol or ethinyl estradiol; and the fatty acid comprises oleic acid, linoleic acid, linolenic acid, stearic acid, palmitic acid, palmitoleic acid, arachidonic acid, eicosenoic acid, docosenoic acid, or tetracosenoic acid. Also, when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.

A method for reducing body weight in a mammal comprising administering a composition comprising (a) a therapeutically effective amount of oleoyl-estrone and (b) a therapeutically effective amount of a beta-3 adrenergic agonist, such as, for example, CL-316243.

In any of these compositions or methods of the present invention, the fatty-acid ester may be substantially pure. Also, the estrogen may comprise estrone, diethylstilbestrol, estriol, estradiol or ethinyl estradiol. In a preferred embodiment, the estrogen comprises estrone. In another embodiment, the estrogen derivative in any of these compositions or methods comprises 2-hydroxyestrone or 2-hydroxy-β-estradiol. In one embodiment, the fatty acid comprises oleic acid, linoleic acid, linolenic acid, stearic acid, palmitic acid, palmitoleic acid, arachidonic acid, eicosenoic acid, docosenoic acid, or tetracosenoic acid. In a preferred embodiment of any of these compositions or methods, the fatty acid comprises oleic acid. In another embodiment, where the fatty acid includes an acyl group; and the estrogen is steroidal and has a steroid ring system with a C-3 position and a hydroxyl group at the C-3 position, the acyl group of the fatty acid is attached to the hydroxyl group at the C-3 position of the steroid ring system in the fatty-acid ester. In an additional embodiment of any of these compositions or methods, the fatty-acid ester comprises a fatty-acid monoester. In a preferred embodiment, the fatty-acid monoester comprises oleoyl-estrone. In one embodiment of any of the compositions or methods, reducing body weight comprises treating obesity or overweight.

In one embodiment of any of these compositions or methods, the therapeutically effective amount of the fatty-acid ester of an estrogen or an estrogen derivative and a fatty acid comprises an amount of about 0.0001 mg/kg/day to about 1000 mg/kg/day. In another embodiment, the therapeutically effective amount of the fatty-acid ester comprises an amount of about 0.001 mg/kg/day to about 200 mg/kg/day. Preferably, the therapeutically effective amount of the fatty-acid ester comprises an amount of about 50 mg/kg/day to about 200 mg/kg/day. In another embodiment, the therapeutically effective amount of the thermogenic compound comprises an amount of about 0.0001 mg/kg/day to about 1000 mg/kg/day. Preferably, the therapeutically effective amount of the thermogenic compound comprises an amount of about 0.001 mg/kg/day to about 200 mg/kg/day.

In one embodiment, the composition or method includes oleoyl-estrone in an amount of about 0.0001 mg/kg/day to about 1000 mg/kg/day. Preferably, the oleoyl-estrone is present in an amount of about 50 mg/kg/day to about 200 mg/kg/day. In another embodiment, a composition or method includes a beta-3 adrenergic agonist in an amount of about 0.0001 mg/kg/day to about 1000 mg/kg/day. In a preferred embodiment, the beta-3 adrenergic agonist is present in an amount of about 0.001 mg/kg/day to about 200 mg/kg/day. In another embodiment, the beta-3 adrenergic agonist is CL-316243, which is present in an amount of about 0.0001 mg/kg/day to about 1000 mg/kg/day.

In one embodiment, the thermogenic compound in any of these compositions or methods comprises a beta-3 adrenergic agonist. In another embodiment, the beta-3 adrenergic agonist comprises CL-316243, AJ-9677, BMS-187413, BMS-187257, BRL-26830A, BRL-28410, BRL-35135, BRL-37344, carazolol, CGP-12177, CL-316243 (BTA-243), CP-114271 (UL-TG-307), CP-331679, FR-149175, FR-165914, L-739574, L-742791, L-749372, L-750355, L-755507, LY-79771, RO-168714, RO-402148, SB-206606, SB-226552, SM-11044, SR-58611A, SR-59062A, trecadrine, TL-TG-307, ZD-2079, or ZD-7114 (ICI-D7114). Any of the compositions of the present invention may further comprise at least one pharmaceutically acceptable carrier. For example, if a first composition and a second composition are administered, the first composition and the second composition may each further comprise at least one pharmaceutically acceptable carrier.

In another embodiment of any of the compositions or methods of the present invention, the fatty-acid ester or thermogenic compound is administered by oral, anal, vaginal, topical, transdermal, intravenous, intramuscular, or subcutaneous administration.

In one embodiment, the fatty-acid ester and the thermogenic compound are administered to a mammal in a single composition comprising the fatty-acid ester and the thermogenic compound. In another embodiment, a therapeutically amount of the fatty-acid ester of an estrogen or an estrogen derivative and a fatty acid is administered in a first composition, and a therapeutically effective amount of the thermogenic compound is administered in a second composition, wherein the first composition or the second composition is administered by oral, anal, vaginal, topical, transdermal, intravenous, intramuscular, or subcutaneous administration.

In another embodiment, the first composition and the second composition are administered to a mammal sequentially. Further, the first composition can be administered before the second composition. In one embodiment, the second composition is administered before the first composition. In an additional embodiment, the first composition and the second composition are administered to a mammal at about the same time.

In one embodiment, a composition for reducing body weight in a mammal comprises a therapeutically effective amount of a fatty-acid ester of an estrogen or an estrogen derivative and a fatty acid; and a therapeutically effective amount of a thermogenic compound, wherein the fatty-acid ester is incorporated into a first liposome and the serotonin reuptake-inhibiting compound is incorporated into a second liposome. In another embodiment, a composition comprises a suspension of the first or second liposome. In an additional embodiment, the liposome suspension is obtainable by addition of soy oil and egg phospholipids.

In another embodiment, a method for reducing body weight in a mammal comprises administering a composition comprising a therapeutically effective amount of a fatty-acid ester of an estrogen or an estrogen derivative and a fatty acid; and a therapeutically effective amount of a thermogenic compound, wherein the fatty-acid ester is incorporated into a first liposome and the thermogenic compound is incorporated into a second liposome. In one embodiment, the method comprises administering a suspension of the first or second liposome. Additionally, the liposome suspension can be obtained by addition of soy oil and egg phospholipids.

### BRIEF DESCRIPTION OF THE DRAWINGS

FIG. 1 is a table showing the body weight change and composition of male Wistar rats treated with oleoyl-estrone and a beta-3 adrenergic agonist.

FIG. 2 is a table showing the energy balance of male Wistar rats treated with oleoyl-estrone and a beta-3 adrenergic agonist.

FIG. 3 is a table showing the plasma composition of Wistar rats treated with oleoyl-estrone and a beta-3 adrenergic agonist.

### DETAILED DESCRIPTION OF THE INVENTION

In one embodiment, the present invention is directed to a method for reducing body weight in a mammal comprising administering a therapeutically effective amount of a fatty-acid ester of an estrogen or an estrogen derivative and a fatty acid; and administering a therapeutically effective amount of a thermogenic compound. This embodiment may include sequential or simultaneous administration of the fatty-acid ester and the thermogenic compound. If administered sequentially, the fatty-acid ester can be administered before or after the thermogenic compound is administered. Additionally, the fatty-acid ester and the thermogenic compound can be in the same or separate compositions prior to administration.

The present invention is also directed towards a method for reducing body weight in a mammal comprising administering a first composition comprising a therapeutically effective amount of a fatty-acid ester of an estrogen or an estrogen derivative and a fatty acid; and administering a second composition comprising a therapeutically effective amount of a thermogenic compound. Prior to administration, the fatty-acid ester and the thermogenic compound are in separate compositions. However, the order of administration does not matter. For instance, the fatty-acid ester can be administered prior to administration of the thermogenic compound. Alternatively, administration of the thermogenic compound can precede administration of the fatty-acid ester.

In another embodiment, the present invention is directed towards a composition for reducing body weight in a mammal comprising a therapeutically effective amount of a fatty-acid ester of an estrogen or an estrogen derivative and a fatty acid; and a therapeutically effective amount of a thermogenic compound.

The present invention is also directed towards a method for reducing body weight in a mammal comprising administering a composition comprising a therapeutically effective amount of a fatty-acid ester of an estrogen or an estrogen derivative and a fatty acid; and a therapeutically effective amount of a thermogenic compound.

The present invention includes compositions comprising an effective amount of a fatty-acid ester of an estrogen or an estrogen derivative. In some embodiments, the fatty-acid ester is substantially pure. In some embodiments, the fatty acid can comprise, for instance, oleic acid, linoleic acid, linolenic acid, stearic acid, palmitic acid, palmitoleic acid, arachidonic acid, eicosenoic acid, docosenoic acid, or tetracosenoic acid. Preferably, the fatty-acid comprises oleic acid.

In some embodiments, the estrogen comprises estrone, i.e. 3-hydroxyestra-1,3,5(10)-trien-17-one; diethylstilbestrol, i.e. 4,4′-(1,2-diethyl-1,2-ethenediyl)-bisphenol; estriol, i.e. estra-1,3,5(10)triene-3,16,17-triol, ethinyl estradiol, i.e. 19-nor-17a-pregna-1,3,5(110)-trien-20-yne-3,17-diol; or estradiol. Further, in some embodiments, the estrogen derivative comprises 2 hydroxyestrone or 2 hydroxy-β-estradiol. Preferably, the estrogen comprises an estrone.

The fatty-acid ester of estrogen or an estrogen derivative preferably comprises a fatty-acid monoester, such as, for examples, estrone monooleate (“oleoyl-estrone”), diethylstilbestrol monooleate, estrone monoeicosenoate or diethylstilbestrol monoeicosenoate. Oleoyl-estrone or estrone monooleate has the chemical formula: 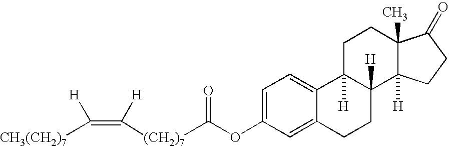
which is also known as [3(Z)]-3-[(1-oxo-9-octadecenyl)oxy]-estra-1,3,5(10)-trien-17-one. In some embodiments, the fatty-acid includes an acyl group and the estrogen is steroidal and has a steroid ring system with a C-3 position and a hydroxyl group at the C-3 position. In these embodiments, the acyl group of the fatty acid is attached to the hydroxyl group at the C-3 position of the steroid ring system in the fatty-acid ester.

In the present invention, the fatty-acid ester is administered along with a thermogenic compound, which includes but is not limited to adrenergic agonists, such as a beta-3 agonist to lower the body weight in a mammal. Suitable beta-3 adrenergic agonists include, for example, CL-316243, AJ-9677, BMS-187413, BMS-187257, BRL-26830A, BRL-28410, BRL-35135, BRL-37344, carazolol, CGP-12177, CL-316243 (BTA-243), CP-114271 (UL-TG-307), CP-331679, FR-149175, FR-165914, L-739574, L-742791, L-749372, L-750355, L-755507, LY-79771, RO-168714, RO-402148, SB-206606, SB-226552, SM-11044, SR-58611A, SR-59062A, trecadrine, TL-TG-307, ZD-2079, and ZD-7114 (ICI-D7114).

The daily dose range of each compound (i.e.-fatty-acid ester or thermogenic compound) in the composition is dependent upon a number of factors, including, the nature of the severity of the condition to be treated, the particular compound in the composition, the route of administration and the age, weight, and response of the individual patient. The daily dose of the fatty-acid ester can generally range from about 0.0001 mg/kg to about 1000 mg/kg, preferably from about 0.001 mg/kg to about 200 mg/kg body weight of a patient in single or separate doses. In some cases it may be necessary to use dosages outside of these ranges. More preferably, the fatty-acid ester is administered in an amount of about 50 mg/kg/day to about 200 mg/kg/day.

The daily dose of the thermogenic compound can generally range from about 0.0001 mg/kg/day to about 1000 mg/kg/day, preferably from about 0.001 mg/kg/day to about 200 mg/kg/day in single or separate doses. In some cases it may be necessary to use dosages outside of these ranges.

In an embodiment where the fatty-acid ester and the thermogenic compound are administered in separate compositions, the appropriate dose of the fatty-acid ester can be about 50 mg/kg/day to about 200 mg/kg/day and the appropriate dose of the thermogenic compound can be about 0.0001 mg/kg/day to about 1000 mg/kg/day.

In an embodiment where the fatty-acid ester and the thermogenic compound are administered together in the same composition, the appropriate dose of the fatty-acid ester is about 50 mg/kg/day to about 200 mg/kg/day and the appropriate dose of the thermogenic compound is about 0.0001 mg/kg/day to about 1000 mg/kg/day.

In the methods and compositions of the present invention, the fatty-acid ester and the thermogenic compound can be administered in separate compositions or in a single composition. Whether they are administered separately or in one composition, each composition is preferably pharmaceutically suitable for administration. The pharmaceutical compositions may be manufactured by means of conventional mixing, dissolving, granulating, dragee-making, levigating, emulsifying, encapsulating, entrapping or lyophilizing processes.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutically acceptable carriers include water, salt solutions, alcohol, silicone, waxes, petroleum jelly, vegetable oil, peanut oil, soybean oil, mineral oil, sesame oil, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, bydroxymethyl-cellulose, polyvinylpyrrolidone, and the like. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The compositions can take the form of solutions, suspensions, emulsions, tablets, pills, capsules, powders, sustained-release formulations and the like, depending on its intended route of administration. Examples of routes of administration include parenteral (e.g., subcutaneous, intramuscular, intraorbital, intracapsular, intraspinal, intrastemal, intravenous, intradermal, intraperitoneal, intraportal, intra-arterial, intrathecal, transmucosal, intra-articular, and intrapleural,), transdermal (i.e., topical), epidural, and mucosal (e.g., intranasal) injection or infusion, as well as oral, inhalation, pulmonary, and rectal administration.

For parenteral administrations, the composition comprises one or more of the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic.

For topical administration, the compositions may be formulated as solutions, gels, ointments, creams, suspensions, etc. as are well-known in the art.

For injection, the compositions may be formulated in solutions, preferably in physiologically compatible buffers such as Hanks's solution, Ringer's solution, or physiological saline buffer. The solution may contain formulatory agents such as suspending, stabilizing and/or dispersing agents. In a preferred embodiment, the compositions are formulated in sterile solutions.

For intravenous administration, suitable carriers include physiological saline, bacteriostatic water, Cremophor® EL (BASF; Parsippany, N.J.) or phosphate buffered saline (PBS). In all cases, the composition must be sterile and should be fluid to the extent that easy injectability with a syringe. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

For transdermal administration, the composition may be formulated into ointments, salves, gels, or creams as generally known in the art. The compounds can also be prepared in the form of suppositories (e.g., with conventional suppository bases such as cocoa butter and other glycerides) or retention enemas for rectal delivery.

For oral administration, the compositions may be formulated as tablets, pills, dragees, troches, capsules, liquids, gels, syrups, slurries, suspensions and the like, for oral ingestion by a patient to be treated. For oral solid formulations such as, for example, powders, capsules and tablets, suitable excipients include fillers such as sugars, e.g., lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); fats and oils; granulating agents; and binding agents such as microcrystalline cellulose, gum tragacanth or gelatin; an excipient, such as starch or lactose; a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations, the compositions may be formulated as mouthwash, suspensions, elixirs and solutions, suitable carriers, excipients or diluents include water, glycols, oils, alcohols, etc. Additionally, flavoring agents, preservatives, coloring agents and the like may be added.

For administration by inhalation, the compositions may be formulated as an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator may be formulated containing a powder mix of the composition and a suitable powder base such as lactose or starch.

The composition may also be formulated in rectal or vaginal compositions such as suppositories or retention enemas, e.g., containing conventional suppository bases such as cocoa butter or other glycerides.

In addition to the formulations described previously, the composition may also be formulated as a depot preparation. Such long acting formulations may be administered by implantation (for example subcutaneously or intramuscularly) or by intramuscular injection. Thus, for example, the therapeutic agents may be formulated with suitable polymeric or hydrophobic materials (for example as an emulsion in an acceptable oil) or ion exchange resins, or as sparingly soluble derivatives, for example, as a sparingly soluble salt.

Additionally, the composition may be delivered using a sustained-release system, such as semi-permeable matrices of solid polymers containing the composition. Various forms of sustained-release materials have been established and are well known by those skilled in the art. Sustained-release capsules may, depending on their chemical nature, release the composition for a few hours, days, weeks, months, up to over 100 days. Depending on the chemical nature and the biological stability of the composition, additional strategies for stabilization may be employed.

In one embodiment, the compositions are incorporated into liposomes. Such compositions can be incorporated into liposomes using known techniques.

In another embodiment, the delivery system for the compositions of this invention is the continuous intravenous injection of the composition integrated in a lipidic suspension. In a particular embodiment, this lipidic suspension is a lipoprotein suspension. In another embodiment, the lipidic suspension is a liposome suspension, preferably obtained by addition of soy oil and egg phospholipids. The formulation should be substantially isotonic with the blood of the treated mammal, and it should contain the composition in the form of a stable lipidic suspension, i.e., in the form of finally divided particles incorporated in suspended microdrops with protecting layers of lipids, these lipids being of lipoproteins or of any common constituents of liposomes.

An example of a preparation of the above-mentioned preferred formulation comprises the steps of: a) mixing a lipidic solution of the composition with an isotonic aqueous phase; and b) sonicating the obtained mixture until a stable suspension is reached. Common techniques of liposome preparations can be used for this preparation. The formulation can be commercially distributed either ready-for-use or in a concentrated form. It can also be distributed with the composition and the lipids separated, as a kit-of-parts.

### EXAMPLE

Forty-five day-old male Wistar rats were used. The rats were kept in collective cages in a light cycle, temperature, and humidity-controlled environment. They were fed tap water and a self-selected cafeteria diet (containing an excess of tasty, energy-dense foods, as well as standard chow) for 45 additional days. Then they were subjected to a 5-day diet adaptation period, in which they had rat chow as the sole food available. At this point, the rats weighed 350-370 g and had significant amounts of body fat. Maintaining the availability of rat chow (daily consumption by the group was recorded) and tap water, body weight was recorded daily. The animals were given a daily gavage of either 0.2 mL of sunflower oil or the same vehicle containing oleoyl-estrone to a daily dose of 10 micromol/kg of body weight.

This standard feeding and drug administration schedule was complemented by treating one half of the animals receiving only oil and one half of the animals receiving oil and oleoyl-estrone with a daily dose of 1 mg/kg of CL-316243, a beta-3 adrenergic agonist. This drug was continuously administered by means of subcutaneous infusion using Alzet osmotic minipumps (model 2002; 0.5 microliter/h) loaded with drug dissolved in saline. The minipumps were inserted subcutaneously in the dorsal lumbar area of the experimental animals by means of a short cut in the skin. The pumps were tested before their implantation, and the fluid remaining in the minipumps was later measured in order to check the effectiveness of the infusion.

The treatment was maintained up to 10 days, when the animals were killed by decapitation, and their blood and carcass were used for analyses.

Plasma was separated from the blood. The plasma was preserved frozen until its utilization for the estimation of glucose, cholesterol, triacylglycerol, free fatty acids, and insulin levels using standard methods, as well as for the estimation of alanine- and aspartate-transaminase activities.

The rat corpses, partially exsanguinated were dissected. The stomach and intestines were cleaned of their contents. The remaining carcass was sealed into polyethylene bags, autoclaved and blended to a fine paste. This paste was analyzed for lipid content by extraction using trichloromethane/methanol and corrected by its water content. Lipid content in the paste was referred to whole in vivo body weight for comparison.

Samples of the rat paste were dried and used for the estimation of their caloric content using a bomb calorimeter. The initial values for lipid content were calculated from the corresponding in vivo body weights at the beginning of the experiment and applying to all the animals the mean percentage of lipid content found in the control (vehicle only) group at the end of the experiment. This same procedure was used to determine the overall energy content of the carcasses by using the experimentally found energy content of the rats in the control group. Likewise, the energy content of the pellet diet was also measured and used to estimate the energy intake of the animals.

Energy expenditure (Ee) was estimated as the difference between energy intake (Ei) and accrual (Ea), since: Ei=Ee±Ea. Energy data are expressed in W (J/s) in order to make the data comparable within the time frame.

Results:

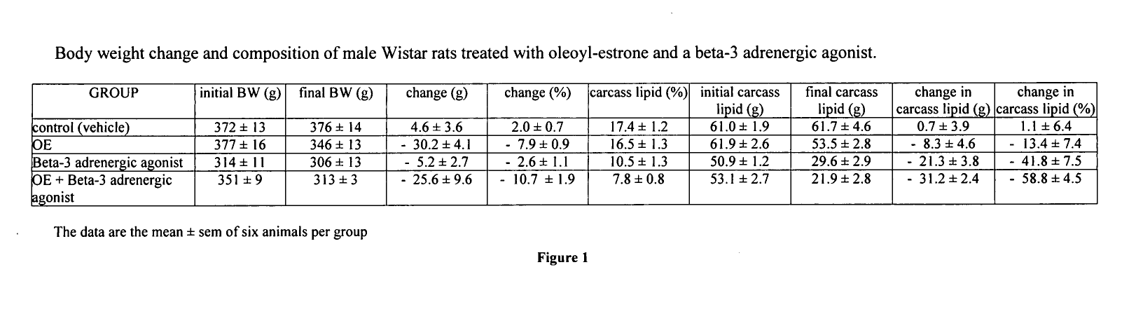

FIG. 1 shows the body weight and lipid changes experienced by the animals in the 10-day period of treatment. Controls barely changed their body weight and lipid content. Oleoyl-estrone treatment induced a loss of body weight of about 8%, mainly derived from lipid stores (loss of 13%). The beta-3 agonist induced a minimal change in body weight (less than 3%), but the lipolytic effects were massive (loss of 42% of lipids). In combination with oleoyl-estrone, the loss of body weight was almost 11% and the loss of lipid increased to 59%, more than obtained by adding up the lipid lost by each single drug treatment alone.

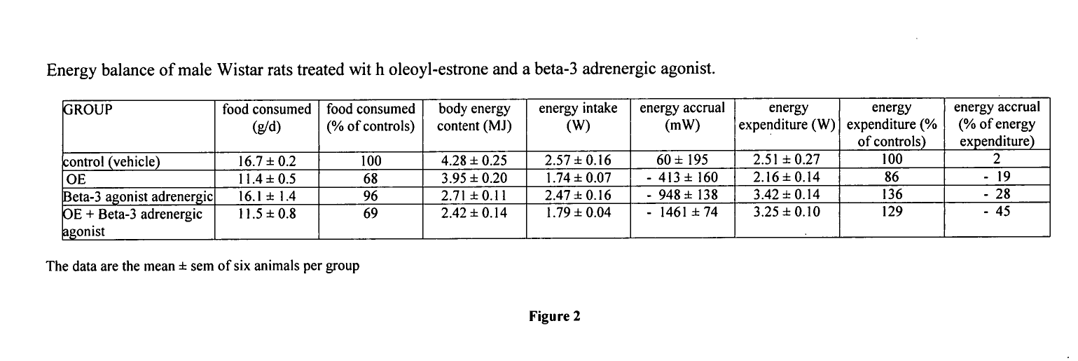

These data were confirmed by the analysis of crude energy content (FIG. 2). The carcass energy content of control group was the highest, followed by the oleoyl-estrone group, then the beta-3 adrenergic agonist group, and, finally, with minimal energy content, the group receiving oleoyl-estrone plus the beta-3 adrenergic agonist.

The loss of energy from the internal stores was very high in all drug-treated animals, following the same pattern found for body weight and lipid content. The loss of body energy experienced by the beta-3 agonist-treated group was more than double (821 kJ) that of the group treated with oleoyl-estrone (354 kJ). The group receiving the combination of both drugs lost even more body energy than the sum of both drugs administered individually (1261 kJ).

These effects were accomplished, in part, due to a marked decrease in food consumption in the oleoyl-estrone (down by 32%), and in the oleoyl-estrone plus beta-3 agonist (down by 31%) groups. The beta-3 agonist group did not show a significant loss of appetite, since they ate approximately the same amount as the control group.

The changes in energy expenditure were, however, considerable in the beta-3 agonist-treated animals, since it was higher than that of the control group by more than one-third. The slight decrease in energy expenditure in the oleoyl-estrone group was largely compensated in the group receiving both drugs. The contribution of internal reserves to fuel the energy expenditure in the oleoyl-estrone group was 19%. This contribution among the beta-3 agonist group was much larger, 28%, and the contribution resulting from the combination of both drugs was a staggering 45%.

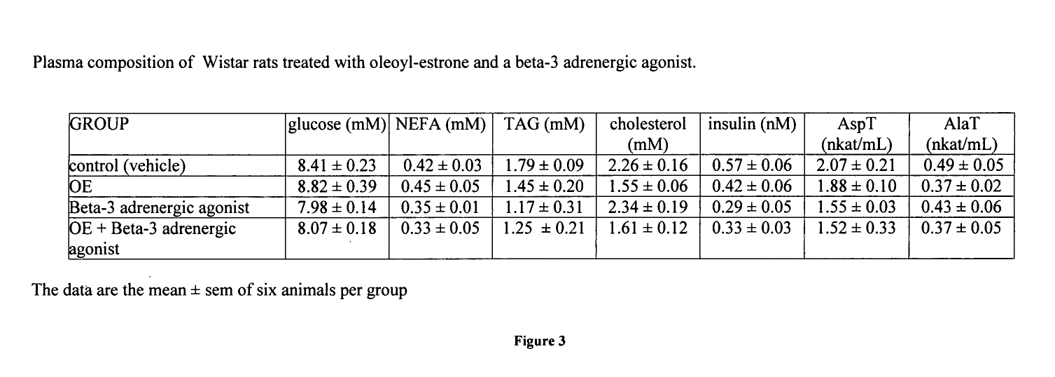

FIG. 3 presents the plasma composition of the rats studied. Treatment with either agent or both combined did not result in significant changes in glucose, non-esterified fatty acids (NEFA), triacylglycerols, insulin or transaminase activities. Total cholesterol, however, showed a marked decrease versus controls in all groups receiving oleoyl-estrone.

The combination of oleoyl-estrone and a thermogenic beta-3 adrenergic agonist at their standard doses resulted in a synergistic effect on the loss of body energy in overweight male rats.

The combination of oleoyl-estrone and a beta-3 adrenergic agonist resulted in the same effects on food,intake than those provoked by oleoyl-estrone alone (decreases of 31-32% in both cases). Additionally, the increase in energy expenditure in all rats treated with the beta-3 agonist was similar (136% and 129%), suggesting that oleoyl-estrone does not increase energy expenditure. Nevertheless, oleoyl-estrone prevents the drop in energy expenditure that occurs with decreased food intake. The changes in body weight and lipid content agree with the overall changes in energy budget described. As a consequence of the synergistic effect of both oleoyl-estrone and a beta-3 agonist, the utilization of internal energy stores is enhanced by the combination of a decrease in energy intake and an increase in energy expenditure. In any case, this composition does not affect the glucose or plasma lipid homeostasis in a significant way. The extreme drawing of energy from fat stores is akin to absolute starvation in its intensity. Nevertheless, no deep changes in glycemia or insulinaemia were observed. Lipid mobilization did not result in increased circulating lipids, since these were maintained. However, the decrease in circulating cholesterol points towards a faster lipoprotein turnover fueled by peripheral lipid oxidation. Furthermore, the unchanged transaminase levels hint to a lack of overall hepatic damage in spite of the intense mobilization of substrates carried across this organ. The maintained glycaemia is a key element in the maintenance of body energy homeostasis, but also a signal of satiety, which can help explain the low food intake observed despite dwindling fat reserves.

The combination of oleoyl-estrone (decreases appetite, maintains energy expenditure, increases lipid mobilization, turnover and oxidation, maintains glycaemia, reduces insulin resistance and decreases hypercholesterolemia) and a beta-3 adrenergic agonist (enhances energy expenditure and thermogenesis) results in the addition of a number of these effects, suggesting that: a) their mode of action is not coincidental, b) their mode of action is not mutually excluding; c) their combination may induce a synergistic enhancement of both effects; and d) in spite of the severe drainage of energy, no apparent ill-effects were observed in the animals subjected to combined treatment with oleoyl-estrone and a beta-3 adrenergic agonist.

While the foregoing description and drawings may represent preferred embodiments of the present invention, it should be understood that various additions, modifications, and substitutions may be made therein without departing from the spirit and scope of the present invention as defined in the accompanying claims. In particular, it will be clear to those skilled in the art that the present invention may be embodied in other specific forms, structures, arrangements, and proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims and not limited to the foregoing description. Furthermore, all references mentioned herein are incorporated by reference in their entirety for all purposes.

1-52. (canceled)

53. A composition for reducing body weight in a mammal comprising (a) a therapeutically effective amount of a fatty-acid ester of an estrogen or an estrogen derivative and a fatty acid; and (b) a therapeutically effective amount of a thermogenic compound.

54. The composition of claim 53, wherein the fatty-acid ester is substantially pure.

55. The composition of claim 53, wherein the estrogen comprises estrone, diethylstilbestrol, estriol, estradiol or ethinyl estradiol.

56. The composition of claim 53, wherein the estrogen comprises estrone.

57. The composition of claim 53, wherein the estrogen derivative comprises 2-hydroxyestrone or 2-hydroxy-β-estradiol.

58. The composition of claim 53, wherein the fatty acid comprises oleic acid, linoleic acid, linolenic acid, stearic acid, palmitic acid, palmitoleic acid, arachidonic acid, eicosenoic acid, docosenoic acid, or tetracosenoic acid.

59. The composition of claim 53, wherein the fatty acid comprises oleic acid.

60. The composition of claim 53, wherein the fatty acid includes an acyl group; and wherein when the estrogen is steroidal and has a steroid ring system with a C-3 position and a hydroxyl group at the C-3 position, the acyl group of the fatty acid is attached to the hydroxyl group at the C-3 position of the steroid ring system in the fatty-acid ester.

61. The composition of claim 53, wherein the fatty-acid ester comprises a fatty-acid monoester.

62. The method of claim 61, wherein the fatty-acid monoester comprises oleoyl-estrone.

63. The composition of claim 53, wherein the fatty-acid ester is present in an amount of about 0.0001 mg/kg/day to about 1000 mg/kg/day.

64. The composition of claim 63, wherein the fatty-acid ester is present in an amount of about 0.001 mg/kg/day to about 200 mg/kg/day.

65. The composition of claim 63, wherein the fatty-acid ester is present in an amount of about 50 mg/kg/day to about 200 mg/kg/day.

66. The composition of claim 62, wherein oleoyl-estrone is present in an amount of about 0.0001 mg/kg/day to about 1000 mg/kg/day.

67. The composition of claim 66, wherein oleoyl-estrone is present in an amount of about 50 mg/kg/day to about 200 mg/kg/day.

68. The composition of claim 53, wherein the thermogenic compound comprises a beta-3 adrenergic agonist.

69. The composition of claim 53, wherein the beta-3 adrenergic agonist comprises CL-316243, AJ-9677, BMS-187413, BMS-187257, BRL-26830A, BRL-28410, BRL-35135, BRL-37344, carazolol, CGP-12177, CL-316243 (BTA-243), CP-114271 (UL-TG-307), CP-331679, FR-149175, FR-165914, L-739574, L-742791, L-749372, L-750355, L-755507, LY-79771, RO-168714, RO-402148, SB-206606, SB-226552, SM-11044, SR-58611A, SR-59062A, trecadrine, TL-TG-307, ZD-2079, or ZD-7114 (ICI-D7114).

70. The composition of claim 53, wherein the thermogenic compound is present in an amount of about 0.0001 mg/kg/day to about 1000 mg/kg/day.

71. The composition of claim 70, wherein the thermogenic compound is present in an amount of about 0.001 mg/kg/day to about 200 mg/kg/day.

72. The composition of claim 68, wherein the beta-3 adrenergic agonist is present in an amount of about 0.0001 mg/kg/day to about 1000 mg/kg/day.

73. The composition of claim 72, wherein the beta-3 adrenergic agonist is present in an amount of about 0.001 mg/kg/day to about 200 mg/kg/day.

74. The composition of claim 69, wherein CL-316243 is present in an amount of about 0.0001 mg/kg/day to about 1000 mg/kg/day.

75. The composition of claim 53 further comprising at least one pharmaceutically acceptable carrier.

76. The composition of claim 53, wherein the fatty-acid ester is incorporated into a first liposome and the thermogenic compound is incorporated into a second liposome.

77. A composition comprising a suspension of the first or second liposome of claim 76.

78. The composition of claim 77, wherein the liposome suspension is obtainable by addition of soy oil and egg phospholipids.

79. A composition for reducing body weight in a mammal comprising (a) a therapeutically effective amount of a substantially pure fatty-acid monoester of an estrogen or an estrogen derivative and a fatty acid, said fatty acid including an acyl group; wherein the estrogen comprises estrone, diethylstilbestrol, estriol or ethinyl estradiol; and wherein the fatty acid comprises oleic acid, linoleic acid, linolenic acid, stearic acid, palmitic acid, palmitoleic acid, arachidonic acid, eicosenoic acid, docosenoic acid, or tetracosenoic acid; and with the proviso that, when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester and (b) a therapeutically effective amount of a thermogenic compound.

80. A composition for reducing body weight in a mammal comprising (a) a therapeutically effective amount of oleoyl-estrone and (b) a therapeutically effective amount of a beta-3 adrenergic agonist.

81. A composition for reducing body weight in a mammal comprising (a) a therapeutically effective amount of oleoyl-estrone and (b) a therapeutically effective amount of CL-316243.

82-110. (canceled)
